(19) United States
(12) Patent Application Publication
(10) Pub. No.: US 2011/0065811 A1
(43) Pub. Date: Mar. 17, 2011

(54) Ethanolamine Glyceryl Ethers

(75) Inventors: Franz-Xaver Scherl, Burgkirchen (DE); Joachim Hess, Hofheim (DE); Ralf Zerrer, Karlstein (DE); Christian Sowa, Neustadt/Weinstrasse (DE)

(73) Assignee: CLARIANT INTERNATIONAL LTD., 4132 Muttenz (CH)

(21) Appl. No.: 12/293,671

(22) PCT Filed: Mar. 15, 2007
(86) PCT No.: PCT/EP07/02273

§ 371 Date: Oct. 20, 2008

(30) Foreign Application Priority Data
Mar. 22, 2006 (DE) 102006013058.8
Mar. 15, 2007 (EP) PCT/EP2007/002273

(51) Int. Cl. B01F 17/16; C07C 215/06

(52) U.S. Cl. 516/9; 564/503

(57) ABSTRACT

Ethanolamine glyceryl ether condensation products of the formula (I) are described,

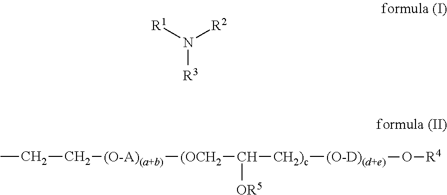

in which R¹, R² and R³ may each independently be the same or different and are each hydrogen, a linear or branched, saturated alkyl chain having from 1 to 20 carbon atoms, a linear or branched unsaturated alkenyl group having from 2 to 20 carbon atoms, —CH₂—CH₂—OH or a radical of the formula (II), with the proviso that at least one of the R¹, R² and R³ radicals is a radical of the formula (II) and at least one further radical of the R¹, R² and R³ radicals is selected from —CH₂—CH₂—OH and radicals of the formula (II), wherein (O-A), (O-D), R⁴, R⁵, a, b, d, and e are as defined in the specification.

The invention relates to ethanolamine glyceryl ether condensation products and their use in dispersions.

Dispersions are substance systems consisting of two or more phases in which one substance (the dispersed or disperse phase) is distributed; i.e. dispersed, in very fine form in another (the dispersing agent or dispersant). Both the dispersing agent and also the substances of the disperse phase may be solid, liquid or gaseous. According to the degree of distribution, a distinction is made between molecularly disperse systems, colloidally disperse systems, finely disperse systems and coarsely disperse systems. Examples of dispersions are suspensions, emulsions, foams, aerosols and smoke. Many industrial products are used in the form of dispersions.

In order to obtain stable dispersions, in particular suspensions or emulsions, interface-active substances (dispersants or emulsifiers) often have to be added which facilitate distribution of the phases, or make it possible in the first place, and counteract gel formation, phase separation, crystallization or sedimentation of the individual components.

The object was therefore to provide novel efficient dispersants and/or emulsifiers which are readily accessible industrially, can be used for a variety of substance systems, but are also toxicologically safe and ecologically compatible.

Surprisingly, it has been found that this object is achieved by special ethanolamine glyceryl ether condensation products. These exhibit in particular excellent interface-active properties, can be readily used in aqueous dispersions, suspension concentrates and suspoemulsions, but also in emulsions and emulsion concentrates, effect high compatibility of the components, for example adjuvants, dispersants, electrolytes etc. with one another and a high suspensibility (suspension power) of the components, and also a stabilization of the phases.

The invention provides ethanolamine glyceryl ether condensation products of the formula (I)

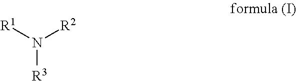

in which
R¹, R² and R³, in each case independently of one another, may be identical or different and
are hydrogen,
are a linear or branched, saturated alkyl chain having 1 to 20, preferably 3 to 20 and particularly preferably 4 to 18, carbon atoms,
are a linear or branched unsaturated alkenyl group having 2 to 20, preferably 3 to 20 and particularly preferably 4 to 18, carbon atoms, are —CH₂—CH₂—OH or
are a radical of the formula (II)

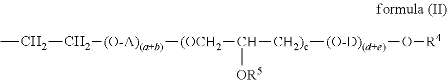

in which
(O-A) is an alkyleneoxy group selected from ethyleneoxy group (EO), propyleneoxy group (PO), butyleneoxy group and/or phenyloxirane group,
a is a number from 0 to 50, preferably 0 to 20, particularly preferably 0 to 10,
b is a number from 0 to 50, preferably 0 to 20, particularly preferably 0 to 10,
   R⁵ is hydrogen and/or
   is (O-A)ₐH, in which (O-A) is an alkyleneoxy group selected from ethyleneoxy group (EO), propyleneoxy group (PO), butyleneoxy group and/or phenyloxirane group and a is a number from 1 to 30, and/or
   is —(CR⁶R⁷)ₐ-phenyl, where R⁶ and R⁷, which may be identical or different, are —H, are an OH group, are a linear or branched (C₁-C₁₀)-alkyl or are a linear or branched (C₂-C₃₀)-alkenyl and a is a number from 1 to 10, and/or
   is —(CR⁶R⁷)ₐ-naphthyl, where R⁶ and R⁷, which may be identical or different, are —H, are an OH group, are a linear or branched (C₁-C₁₀)-alkyl or are a linear or branched (C₂-C₃₀)-alkenyl and a is a number from 1 to 10, and/or
   is a group of the formula (III)

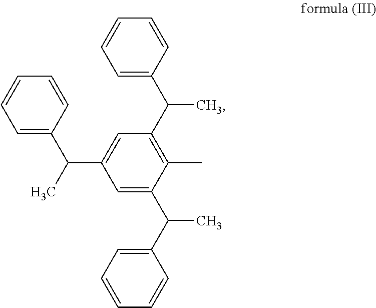

and/or
is R⁸R⁹N—(CH₂)_{b}—, where R⁸ and R⁹, which may be identical or different, are —H, linear or branched (C₁-C₁₀)-alkyl or linear or branched (C₂-C₃₀)-alkenyl and b is a number from 1 to 22, and/or
is HO—(CH₂)_{b}—, where b is a number from 1 to 22, and/or
is —SO₃⁻X⁺, —PO₃²⁻X⁺X′⁺ or —CH₂COO⁻X⁺, where X⁺ and X′⁺ are H⁺, Li⁺, Na⁺, K⁺, Ca²⁺/2, Mg²⁺/2 or N(R⁸)₄⁺ and the R⁸ may be identical or different and are —H or (C₁-C₁₀)-alkyl, preferably (C₁-C₄)-alkyl, and/or
is a group of the formula (IV)

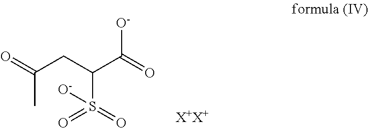

where
X⁺ and X′⁺ are H⁺, Li⁺, Na⁺, K⁺, Ca²⁺/2, Mg²⁺/2 or N(R⁸)₄⁺, and the R⁸ may be identical or different and are —H or (C₁-C₁₀)-alkyl, preferably (C₁-C₄)-alkyl, and/or
is —C(R¹¹)₂—COO⁻X⁺, —CO—R¹²—COO⁻X⁺ or —C(R¹¹)₂C(R¹¹)₂C(R¹¹)₂—N(R¹³)₂, where the R¹¹ may be identical or different and are —H and/or CH₃, R¹² is (C₁-C₁₀)-alkylene or (C₂-C₃₀)-alkenylene, the R¹³ may be identical or different and are (C₁-C₁₀)-alkyl or (C₂-C₃₀)-alkenyl, X⁺ is H⁺, Li⁺, Na⁺, K⁺, Ca²⁺/2, Mg²⁺/2 or N(R⁸)₄⁺, and the R⁸ may be identical or different and are —H or (C₁-C₁₀)-alkyl, preferably (C₁-C₄)-alkyl, and/or
is —C(R¹⁴)₂C(R¹⁴)₂C(R¹⁴)₂—N((GO)_{z}H)₂, where the R¹⁴ may be identical or different and are —H and/or —CH₃, G is —C₂H₄—, —C₃H₆— or —C₄H₈— and z is a number from 1 to 22, and/or
is a group of the formula (V)

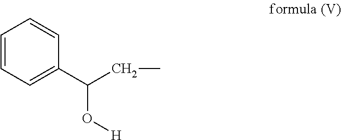

c is a number from 1 to 25, preferably a number from 1 to 20,
(O-D) is an alkyleneoxy group selected from ethyleneoxy group (EO), propyleneoxy group (PO), butyleneoxy group and/or phenyloxirane group,
d is a number from 0 to 50, preferably 0 to 20, particularly preferably 0 to 10,
e is a number from 0 to 50, preferably 0 to 20, particularly preferably 0 to 10, and
R⁴ can have the meaning of R⁵,
   with the proviso that at least one of the radicals R¹, R² and R³ is a radical of the formula (II) and at least one of the other radicals R¹, R² and R³ is selected from —CH₂—CH₂—OH and radicals of the formula (II).

Within the context of the present invention, the alkenyl groups can be mono- or polyunsaturated.

In a preferred embodiment of the invention, at least two of the radicals R¹, R² and R³ are radicals of the formula (II).

In a further preferred embodiment of the invention two of the radicals R¹, R² and R³ are selected from —CH₂—CH₂—OH and radicals of the formula (II). In these compounds, accordingly, one of the radicals R¹, R² and R³ is a radical of the formula (II) and one of the other radicals R¹, R² and R³ is selected from —CH₂—CH₂—OH and radicals of the formula (II). Among these compounds, preference is in turn given to those in which two of the radicals R¹, R² and R³ are radicals of the formula (II). The third of the radicals R¹, R² and R³ is hydrogen or a linear or branched, saturated alkyl chain having 1 to 20, preferably 3 to 20 and particularly preferably 4 to 18, carbon atoms or is a linear or branched unsaturated alkenyl group having 2 to 20, preferably 3 to 20 and particularly preferably 4 to 18, carbon atoms, preferably a linear or branched, saturated alkyl chain having 1 to 20, preferably 3 to 20 and particularly preferably 4 to 18, carbon atoms or is a linear or branched unsaturated alkenyl group having 2 to 20, preferably 3 to 20 and particularly preferably 4 to 18, carbon atoms, and particularly preferably is a linear or branched, saturated alkyl chain having 1 to 20, preferably 3 to 20 and particularly preferably 4 to 18, carbon atoms.

In a further preferred embodiment of the invention all three radicals R¹, R² and R³ are selected from —CH₂—CH₂—OH and radicals of the formula (II). In these compounds, accordingly, at least one of the radicals R¹, R² and R³ is a radical of the formula (II) and the two other radicals R¹, R² and R³ are selected from —CH₂—CH₂—OH and radicals of the formula (II). In a particularly preferred embodiment of the invention, one of the radicals R¹, R² and R³ is a radical of the formula (II) and the two other radicals R¹, R² and R³ are radicals of the formula —CH₂—CH₂—OH. In a further especially preferred embodiment of the invention, two of the radicals R¹, R² and R³ are radicals of the formula (II) and the third of the radicals R¹, R² and R³ is a radical of the formula —CH₂—CH₂—OH. In a further especially preferred embodiment of the invention, all three radicals R¹, R² and R³ are radicals of the formula (II).

A further preferred subject matter of the invention is ethanolamine glyceryl ether condensation products of the formula (I) wherein (O-A) and (O-D) are in each case a uniform alkyleneoxy group, (a+b) is a number from 0 to 20 and (d+e) is a number from 0 to 20.

A further preferred subject matter of the invention is ethanolamine glyceryl ether condensation products of the formula (I) wherein (O-A)_{(a+b)} and (O-D)_{(d+e)} are mixed alkyleneoxy groups of the formula —(O-A¹)ₐ-(O-A²)_{b}- or —(O-D¹)_{d}-(O-D²)ₑ-, which can be arranged randomly or blockwise, and in which a and b are in each case a number from 1 to 30, and d and e are in each case a number from 1 to 20, (O-A¹) and (O-D¹) are an ethyleneoxy unit, and (O-A²) and (O-D²) are a propyleneoxy unit, a butyleneoxy unit, a phenyloxirane unit, or mixtures of these.

A further preferred subject matter of the invention is ethanolamine glyceryl ether condensation products of the formula (I) wherein

the radicals R¹, R² and R³ are identical and are a group of the formula (IIa),

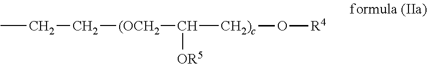

in which
R⁵ is hydrogen,
c is a number from 1 to 25, preferably 1 to 20, particularly preferably 2 to 15, especially preferably 4 to 12, extraordinarily preferably 6 to 10, and
R⁴ is hydrogen.

A further preferred subject matter of the invention is ethanolamine glyceryl ether condensation products of the formula (I) wherein

the radicals R¹, R² and R³ are identical and are a group of the formula (IIa),

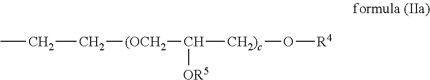

in which
R⁵ is hydrogen,
c is a number from 1 to 25, preferably 1 to 20, particularly preferably 2 to 15, especially preferably 4 to 12, extraordinarily preferably 6 to 10, and
R⁴ is a group of the formula (III)

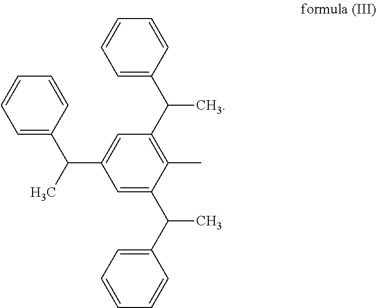

A further preferred subject matter of the invention is ethanolamine glyceryl ether condensation products of the formula (I) wherein

the radicals R¹, R² and R³ are identical and are a group of the formula (IIb)

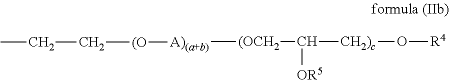

in which
(O-A)_{(a+b)} is a uniform alkyleneoxy group and (a+b) is a number from 1 to 50 and is a mixed alkyleneoxy group of the formula —(O-A¹)ₐ-(O-A²)_{b}-, which can be arranged randomly or blockwise and in which (a+b) is a number from 1 to 50, (O-A¹) is an ethyleneoxy unit and (O-A²) is a propyleneoxy unit, a butyleneoxy unit, a phenyloxirane unit or mixtures of these,
R⁵ is hydrogen,
c is a number from 1 to 25, preferably 1 to 20, particularly preferably 2 to 15, especially preferably 4 to 12, extraordinarily preferably 6 to 10, and
R⁴ is hydrogen.

A further preferred subject matter of the invention is ethanolamine glyceryl ether condensation products of the formula (I) wherein

the radicals R¹, R² and R³ are identical and are a group of the formula (IIc)

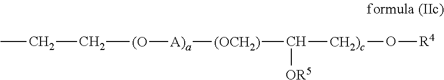

in which
(O-A) is an alkyleneoxy group, preferably a propyleneoxy group,
a is a number from 1 to 50,
R⁵ is hydrogen,
c is a number from 1 to 25, preferably 1 to 20, particularly preferably 2 to 15, especially preferably 4 to 12, extraordinarily preferably 6 to 10, and
R⁴ is hydrogen.

A further preferred subject matter of the invention is ethanolamine glyceryl ether condensation products of the formula (I) wherein
R¹ is an alkyl group having 1 to 20, preferably 3 to 20 and particularly preferably 4 to 18, carbon atoms, and
R² and R₃ are identical and are a group of the formula (IId)

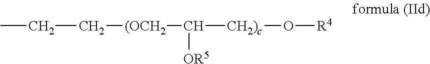

in which
R⁵ is hydrogen,
c is a number from 1 to 25, preferably 1 to 20, particularly preferably 2 to 15, especially preferably 4 to 12, extraordinarily preferably 6 to 10, and
R⁴ is hydrogen.

A further preferred subject matter of the invention is ethanolamine glyceryl ether condensation products of the formula (I) wherein
R¹ is an alkyl group having 1 to 10 carbon atoms, preferably an n-butyl group, and
R² and R₃ are identical and are a group of the formula (IId)

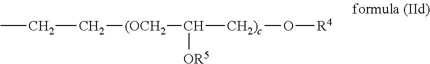

in which
R⁵ is hydrogen,
c is a number from 1 to 25, preferably 1 to 20, particularly preferably 2 to 15, especially preferably 4 to 12, extraordinarily preferably 6 to 10, and
R⁴ is hydrogen.

The ethanolamine glyceryl ether condensation products according to the invention are suitable in an advantageous manner for producing dispersions. A further subject matter of the present invention is dispersions comprising one or more of the ethanolamine glyceryl ether condensation products according to the invention.

The ethanolamine glyceryl ether condensation products according to the invention are additionally suitable in an advantageous manner as interface-active substances. A further subject matter of the present invention is therefore also the use of one or more of the ethanolamine glyceryl ether condensation products according to the invention as interface-active substances.

In a further preferred embodiment of the invention, the ethanolamine glyceryl ether condensation products according to the invention are used as interface-active substances in dispersions.

### EXAMPLES

Preparation examples of glyceryl ethers according to the invention are described below without limiting the invention to these.

### General Preparation Method

### a) Polymerization of Glycerol to Oligoglycerols or Polyglycerols

The polymerization of glycerol to oligoglycerols or polyglycerols takes place in a known manner.

For this, glycerol is heated at 200 to 280° C., preferably 240 to 270° C., in the presence of acidic catalysts, for example HCl, H₂SO₄, sulfonic acids or H₃PO₄ or in the presence of alkaline catalysts, such as sodium hydroxide, potassium hydroxide, alkali metal alkoxides, alkali metal carbonates, alkali metal bicarbonates, in a concentration range from 0.1 to 0.4% by weight of catalyst in a stirred apparatus with water separator and while nitrogen is passed through. While discharging water of condensation, the formation of the oligoglycerol or polyglycerol with an average degree of condensation of from 2 to 100, preferably 3 to 35 glycerol units takes place over the course of 3 to 14 hours. From the OH number it is possible to calculate the average molar mass of the oligo- or polyglycerols.

The ratio of the degree of condensation n to the condensation time during the polymerization of glycerol to oligoglycerols or polyglycerols is shown in table 1.

TABLE 1
Ratio of degree of condensation n to condensation time

| Degree of | OH number | Molar mass | Condensation time |
|---|---|---|---|
| condensation n | [mg KOH/g] | [g/mol] | [hours] |
| 2.0 | 1352 | 166 | 3-4 |
| 3.0 | 1169 | 240 | 3-4 |
| 4.0 | 1072 | 314 | 4-5 |
| 5.0 | 1012 | 388 | 5-7 |
| 6.0 | 971 | 462 | 6-7 |
| 7.0 | 942 | 536 | 7-9 |
| 8.0 | 920 | 610 | 8-9 |
| 9.0 | 902 | 684 | 9-11 |
| 10 | 888 | 758 | 10-11 |
| 11 | 877 | 832 | 11-12 |
| 12 | 867 | 906 | 11-12 |
| 13 | 859 | 980 | 12-13 |
| 14 | 850 | 1056 | 12-13 |
| 15 | 844 | 1130 | 13-14 |

### b) Preparation of Ethanolamine Glyceryl Ethers

To prepare the ethanolamine glyceryl ether condensation products according to the invention, the abovementioned mono-, oligo- or polyglycerols or the corresponding oxalkylated glycerols are combined with nitrogen-containing mono- or polyols and, if appropriate, further comonomers in the desired mixing ratio. Following the addition of 0.1-1.0 mol % (preferably 0.28 mol %) of a basic catalyst (e.g. NaOH, KOH, Na₂CO₃, K₂CO₃, CaCO₃, Li₂CO₃, preferably NaOH, particularly preferably 50% strength by weight aqueous NaOH), the reaction mixture is heated to 220-290° C., preferably 250-270° C., with vigorous stirring and, at this temperature, while azeotropically removing the water which forms, is condensed until the desired degree of condensation is reached. The reaction progress is monitored by regularly determining the OH number.

The compounds according to the invention are characterized by an excellent dispersing power and a high electrolyte stability. Additionally, the glyceryl ethers according to the invention bring about an improvement in the compatibility of hydrophilic and hydrophobic components and in particular an increase in the wetting power and absorption capacity of formulations which comprise these glyceryl ethers.

### Example 1

### Cocondensation of Glycerol/Alkyldiethanolamine

Glycerol and alkyldiethanolamine are initially introduced in the stated molar ratio together with 0.1 to 1.0 mol % of NaOH (based on the amount of glycerol used) in the form of a 50% strength by weight aqueous NaOH solution and, with stirring at 220° C. and light nitrogen blanketing, are subsequently condensed until the calculated target OH number is approximately reached (see table 2). The resulting products are dark brown and solid.

TABLE 2
Ethanolamine glyceryl ether condensation products with
alkyldiethanolamine

| | | | | | Average |
|---|---|---|---|---|---|
| | | | | | molar mass |
| | Glycerol:alkyldiethanolamine | Reaction | OH number | Degree of | from OH |
| Alkyl chain | molar ratio | time | of product | condensation* | number |
| 2-Ethylhexyl | 10:1 | 19 h | 701 mg | 5.0 | 960 g/mol |
| | | | KOH/g | | |
| 2-Ethylhexyl | 30:1 | 17 h | 732 mg | 13 | 2130 g/mol |
| | | | KOH/g | | |
| Coconut | 10:1 | 11 h | 655 mg | 4.4 | 925 g/mol |
| (C₈-C₁₈) | | | KOH/g | | |
| Coconut | 30:1 | 12 h | 729 mg | 20 | 3230 g/mol |
| (C₈-C₁₈) | | | KOH/g | | |

*Degree of condensation of the glycerol per CH₂OH group of the alkydiethanolamine calculated from OH number

### Example 2

### Cocondensation of Glycerol/Triethanolamine

Glycerol and triethanolamine are initially introduced in the stated molar ratio together with 0.1-1.0 mol % of NaOH (based on the amount of glycerol used) in the form of a 50% strength by weight aqueous NaOH solution and, with stirring at 270° C. and light nitrogen blanketing, are condensed until the calculated target OH number is approximately reached (see table 3). The resulting products are dark brown and viscous-flowing to solid.

TABLE 3
Ethanolamine glyceryl ether condensation products with
triethanolamine

| | | | | | Average |
|---|---|---|---|---|---|
| Glycerol:tri- | | | | | molar mass |
| ethanolamine | Reaction | Reaction | OH number | Degree of | from OH |
| molar ratio | time | temperature | of product | condensation* | number |
| 9:1 | 6 h | 270° C. | 820 mg | 3.3 | 883 g/mol |
| | | | KOH/g | | |
| 15:1 | 9 h | 270° C. | 795 mg | 6.1 | 1500 g/mol |
| | | | KOH/g | | |
| 30:1 | 14 h | 270° C. | 781 mg | 10.2 | 2410 g/mol |
| | | | KOH/g | | |
| 45:1 | 18 h | 270° C. | 770 mg | 20.3 | 4660 g/mol |
| | | | KOH/g | | |

*Degree of condensation of the glycerol per CH₂OH group of the triethanolamine calculated from OH number

1. A process for modifying the interfacial activity of a substance, comprising the step of adding at least one ethanolamine glyceryl ether condensation product of the formula (I) 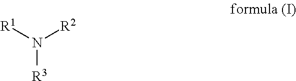
wherein
R¹, R² and R³, in each case independently of one another, may be identical or different and
are hydrogen,
are a linear or branched, saturated alkyl chain having 1 to 20 carbon atoms,
are a linear or branched unsaturated alkenyl group having 2 to 20 carbon atoms,
are —CH₂—CH₂—OH or
are a radical of the formula (II) 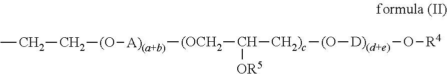
wherein
(O-A) is an alkyleneoxy group selected from ethyleneoxy group (EO), propyleneoxy group (PO), butyleneoxy group and/or phenyloxirane group,
a is a number from 0 to 50,
b is a number from 0 to 50,
R⁵ is hydrogen and/or
is (O-A)ₐH, wherein (O-A) is an alkyleneoxy group selected from the group consisting of: ethyleneoxy group (EO), propyleneoxy group (PO), butyleneoxy group and phenyloxirane group and a is a number from 1 to 30, and/or
is —(CR⁶R⁷)ₐ-phenyl, where R⁶ and R⁷, which may be identical or different, are —H, are an OH group, are a linear or branched (C₁-C₁₀)-alkyl or are a linear or branched (C₂-C₃₀)-alkenyl and a is a number from 1 to 10, and/or
is —(CR⁶R⁷)ₐ-naphthyl, where R⁶ and R⁷, which may be identical or different, are —H, are an OH group, are a linear or branched (C₁-C₁₀)-alkyl or are a linear or branched (C₂-C₃₀)-alkenyl and a is a number from 1 to 10, and/or
is a group of the formula (III) 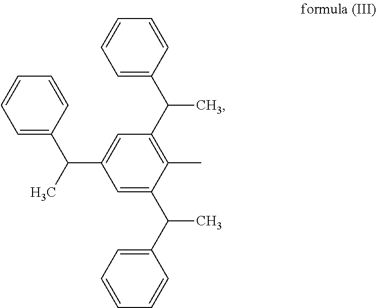
and/or
is R⁸R⁹N—(CH₂)_{b}—, where R⁸ and R⁹, which may be identical or different, are —H, linear or branched (C₁-C₁₀)-alkyl or linear or branched (C₂-C₃₀)-alkenyl and b is a number from 1 to 22, and/or
is HO—(CH₂)_{b}—, where b is a number from 1 to 22, and/or
is —SO₃⁻X⁺, —PO₃²⁻X⁺X′⁺ or —CH₂COO⁻X⁺, where X⁺ and X′⁺ are H⁺, Li⁺, Na⁺, K⁺, Ca²⁺/2, Mg²⁺/2 or N(R⁸)₄⁺ and the R⁸ may be identical or different and are —H or (C₁-C₁₀)-alkyl, and/or
is a group of the formula (IV) 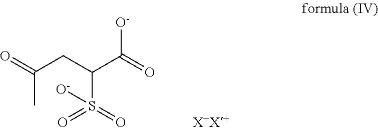
wherein
X⁺ and X′⁺ are H⁺, Li⁺, Na⁺, K⁺, Ca²⁺/2, Mg²⁺/2 or N(R⁸)₄⁺, and the R⁸ may be identical or different and are —H or (C₁-C₁₀)-alkyl, and/or
is —C(R¹¹)₂—COO⁻X⁺, —CO—R¹²—COO⁻X⁺ or
—C(R¹¹)₂C(R¹¹)₂C(R¹¹)₂—N(R¹³)₂, where the R¹¹ may be identical or different and are —H and/or CH₃, R¹² is (C₁-C₁₀)-alkylene or (C₂-C₃₀)-alkenylene, the R¹³ may be identical or different and are (C₁-C₁₀)-alkyl or (C₂-C₃₀)-alkenyl, X⁺ is H⁺, Li⁺, Na⁺, K⁺, Ca²⁺/2, Mg²⁺/2 or N(R⁸)₄⁺, and the R⁸ may be identical or different and are —H or (C₁-C₁₀)-alkyl, and/or
is —C(R¹⁴)₂C(R¹⁴)₂C(R¹⁴)₂—N((GO)_{z}H)₂, where the R¹⁴ may be identical or different and are —H and/or —CH₃, G is —C₂H₄—, —C₃H₆— or —C₄H₈— and z is a number from 1 to 22, and/or
is a group of the formula (V) 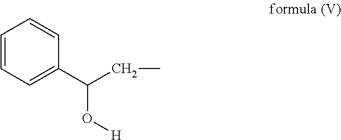
c is a number from 1 to 25,
(O-D) is an alkyleneoxy group selected from ethyleneoxy group (EO), propyleneoxy group (PO), butyleneoxy group and/or phenyloxirane group,
d is a number from 0 to 50,
e is a number from 0 to 50, and
R⁴ can have the meaning of R⁵,
with the proviso that at least one of the radicals R¹, R² and R³ is a radical of the formula (II) and at least one of the other radicals R¹, R² and R³ is selected from —CH₂—CH₂—OH and radicals of the formula (II)
to a dispersion.

2. The process as claimed in claim 1, wherein, in the at least one ethanolamine glyceryl ether condensation product, at least two of the radicals R¹, R² and R³ are radicals of the formula (II).

3. The process as claimed in claim 1, wherein, in the at least one ethanolamine glyceryl ether condensation product, two of the radicals R¹, R² and R³ are radicals of the formula (II).

4. The process as claimed in claim 3, wherein, in the at least one ethanolamine glyceryl ether condensation product, the third of the radicals R¹, R² and R³ is a linear or branched, saturated alkyl chain having 1 to 20, carbon atoms or is a linear or branched unsaturated alkenyl group having 2 to 20 carbon atoms.

5. The process as claimed in claim 4, wherein, in the at least one ethanolamine glyceryl ether condensation product, the third of the radicals R¹, R² and R³ is a linear or branched, saturated alkyl chain having 1 to 20 carbon atoms.

6. The process as claimed in claim 1, wherein, in the at least one ethanolamine glyceryl ether condensation product, all three radicals R¹, R² and R³ are selected from the group consisting of CH₂—H₂—OH and radicals of the formula (II).

7. The process as claimed in claim 6, wherein, in the at least one ethanolamine glyceryl ether condensation product, all three radicals R¹, R² and R³ are radicals of the formula (II).

8. The process as claimed in claim 1, wherein, in the at least one ethanolamine glyceryl ether condensation product, (O-A) and (O-D) are in each case a uniform alkyleneoxy group, (a+b) is a number from 0 to 20 and (d+e) is a number from 0 to 20.

9. The process as claimed in claim 1, wherein, in the at least one ethanolamine glyceryl ether condensation product, (O-A)_{(a+b)} and (O-D)_{(d+e)} are mixed alkyleneoxy groups of the formula —(O-A¹)ₐ-(O-A²)_{b}- or —(O-D¹)_{d}-(O-D²)ₑ-, which may be arranged randomly or blockwise, and in which a and b are in each case a number from 1 to 30, and d and e are in each case a number from 1 to 20, (O-A¹) and (O-D¹) are an ethyleneoxy unit, and (O-A²) and (O-D²) are a propyleneoxy unit, a butyleneoxy unit, a phenyloxirane unit, or mixtures of these.

10. The process as claimed claim 1, wherein, in the at least one ethanolamine glyceryl ether condensation product, the radicals R¹, R² and R³ are identical and are a group of the formula (IIa), 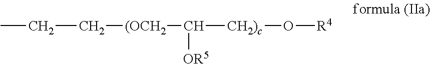
wherein
R⁵ is hydrogen,
c is a number from 1 to 25, and
R⁴ is hydrogen.

11. The process as claimed claim 1, wherein, in the at least one ethanolamine glyceryl ether condensation product, the radicals R¹, R² and R³ are identical and are a group of the formula (IIa), 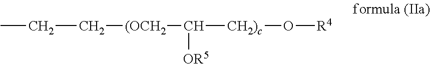
wherein
R⁵ is hydrogen,
c is a number from 1 to 25, and
R⁴ is a group of the formula (III)

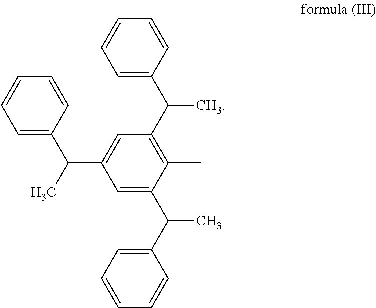

12. The process as claimed claim 1, wherein, in the at least one ethanolamine glyceryl ether condensation product, the radicals R¹, R² and R³ are identical and are a group of the formula (IIb), 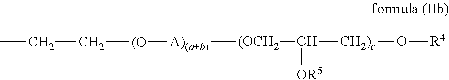
wherein
(O-A)_{(a+b)} is a uniform alkyleneoxy group and (a+b) is a number from 1 to 50 or is a mixed alkyleneoxy group of the formula -(O-A¹)ₐ-(O-A²)_{b}-, which can be arranged randomly or blockwise and in which (a+b) is a number from 1 to 50, (O-A¹) is an ethyleneoxy unit and (O-A²) is a propyleneoxy unit, a butyleneoxy unit, a phenyloxirane unit or mixtures of these,
R⁵ is hydrogen,
c is a number from 1 to 25, and
R⁴ is hydrogen.

13. The process as claimed in claim 1, wherein, in the at least one ethanolamine glyceryl ether condensation products, the radicals R¹, R² and R³ are identical and are a group of the formula (IIc), 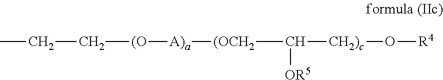
in which
(O-A) is an alkyleneoxy group,
a is a number from 1 to 50,
R⁵ is hydrogen,
c is a number from 1 to 25, and
R⁴ is hydrogen.

14. The process as claimed in claim 1, wherein, in the at least one ethanolamine glyceryl ether condensation product,
R¹ is an alkyl group having 1 to 20, carbon atoms, and
R² and R₃ are identical and are a group of the formula (IId) 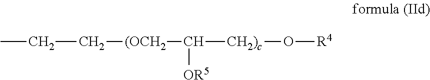
wherein
R⁵ is hydrogen,
c is a number from 1 to 25, and
R⁴ is hydrogen.

15. The process as claimed in claim 1, wherein, in the at least one ethanolamine glyceryl ether condensation product,
R¹ is an alkyl group having 1 to 10 carbon atoms, and
R² and R₃ are identical and are a group of the formula (IId) 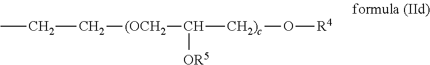
wherein
R⁵ is hydrogen,
c is a number from 1 to 25, and
R⁴ is hydrogen.

16. (canceled)

17. An ethanolamine glyceryl ether condensation product of the formula (I) 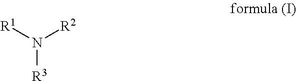
wherein
R¹, R² and R³, in each case independently of one another, may be identical or different and
are hydrogen,
are a linear or branched, saturated alkyl chain having 1 to 20 carbon atoms,
are a linear or branched unsaturated alkenyl group having 2 to 20 carbon atoms,
are —CH₂—CH₂—OH or
are a radical of the formula (II) 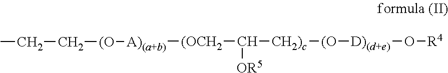
wherein
(O-A) is an alkyleneoxy group selected from the group consisting of: ethyleneoxy group (EO), propyleneoxy group (PO), butyleneoxy group and phenyloxirane group,
a is a number from 0 to 50,
b is a number from 0 to 50,
R⁵ is hydrogen and/or
is (O-A)ₐH, in which (O-A) is an alkyleneoxy group selected from the group consisting of: ethyleneoxy group (EO), propyleneoxy group (PO), butyleneoxy group and phenyloxirane group and a is a number from 1 to 30, and/or
is —(CR⁶R⁷)ₐ-phenyl, where R⁶ and R⁷, which may be identical or different, are —H, are an OH group, are a linear or branched (C₁-C₁₀)-alkyl or are a linear or branched (C₂-C₃₀)-alkenyl and a is a number from 1 to 10, and/or
is —(CR⁶R⁷)ₐ-naphthyl, where R⁶ and R⁷, which may be identical or different, are —H, are an OH group, are a linear or branched (C₁-C₁₀)-alkyl or are a linear or branched (C₂-C₃₀)-alkenyl and a is a number from 1 to 10, and/or is a group of the formula (III) 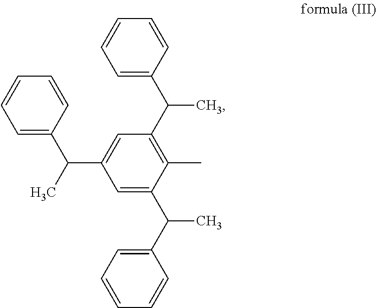
and/or
is R⁸R⁹N—(CH₂)_{b}—, where R⁸ and R⁹, which may be identical or different, are —H, linear or branched (C₁-C₁₀)-alkyl or linear or branched (C₂-C₃₀)-alkenyl and b is a number from 1 to 22, and/or
is HO—(CH₂)_{b}—, where b is a number from 1 to 22, and/or
is —SO₃⁻X⁺, —PO₃²⁻X⁺X′⁺ or —CH₂COO⁻X⁺, where X⁺ and X′⁺ are H⁺, Li⁺, Na⁺, K⁺, Ca²⁺/2, Mg²⁺/2 or N(R⁸)₄⁺ and the R⁸ may be identical or different and are —H or (C₁-C₁₀)-alkyl, and/or
is a group of the formula (IV) 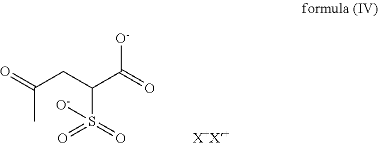
wherein
X⁺ and X′⁺ are H⁺, Li⁺, Na⁺, K⁺, Ca²⁺/2, Mg²⁺/2 or N(R⁸)₄⁺, and the R⁸ may be identical or different and are —H or (C₁-C₁₀)-alkyl, and/or
is —C(R¹¹)₂—COO⁻X⁺, —CO—R¹²—COO⁻X⁺ or —C(R¹¹)₂C(R¹¹)₂C(R¹¹)₂—N(R¹³)₂, where the R¹¹ may be identical or different and are —H and/or CH₃, R¹² is (C₁-C₁₀)-alkylene or (C₂-C₃₀)-alkenylene, the R¹³ may be identical or different and are (C₁-C₁₀)-alkyl or (C₂-C₃₀)-alkenyl, X⁺ is H⁺, Li⁺, Na⁺, K⁺, Ca²⁺/2, Mg²⁺/2 or N(R⁸)₄⁺, and the R⁸ may be identical or different and are —H or (C₁-C₁₀)-alkyl, and/or
is —C(R¹⁴)₂C(R¹⁴)₂C(R¹⁴)₂—N((Go)_{Z}H)₂, where the R¹⁴ may be identical or different and are —H and/or —CH₃, G is —C₂H₄—, —C₃H₆— or —C₄H₈— and z is a number from 1 to 22, and/or
is a group of the formula (V) 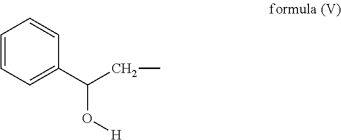
c is a number from 1 to 25,
(O-D) is an alkyleneoxy group selected from the group consisting of: ethyleneoxy group (EO), propyleneoxy group (PO), butyleneoxy group and phenyloxirane group,
d is a number from 0 to 50,
e is a number from 0 to 50, and
R⁴ can have the meaning of R⁵,
with the proviso that at least two of the radicals R¹, R² and R³ are radicals of the formula (II) and at least one of the other radicals R¹, R² and R³ is selected from the group consisting of —CH₂—CH₂—OH and radicals of the formula (II).

18. An ethanolamine glyceryl ether condensation product as claimed in claim 17, wherein two of the radicals R¹, R² and R³ are radicals of the formula (II).

19. An ethanolamine glyceryl ether condensation product as claimed in claim 18, wherein the third of the radicals R¹, R² and R³ is a linear or branched, saturated alkyl chain having 1 to 20, carbon atoms or is a linear or branched unsaturated alkenyl group having 2 to 20, carbon atoms.

20. An ethanolamine glyceryl ether condensation product as claimed in claim 19, wherein the third of the radicals R¹, R² and R³ is a linear or branched, saturated alkyl chain having 1 to 20 carbon atoms.

21. An ethanolamine glyceryl ether condensation product as claimed in claim 17, wherein all three radicals R¹, R² and R³ are selected from the group consisting of CH₂—H₂—OH and radicals of the formula (II).

22. An ethanolamine glyceryl ether condensation product as claimed in claim 21, wherein all three radicals R¹, R² and R³ are radicals of the formula (II).

23. An ethanolamine glyceryl ether condensation product as claimed in claim 17, wherein (O-A) and (O-D) are in each case a uniform alkyleneoxy group, (a+b) is a number from 0 to 20 and (d+e) is a number from 0 to 20.

24. An ethanolamine glyceryl ether condensation product as claimed in claim 17, wherein (O-A)_{(a+b)} and (O-D)_{(d+e)} are mixed alkyleneoxy groups of the formula —(O-A¹)ₐ-(O-A²)_{b}- or —(O-D¹)_{d}-(O-D²)ₑ-, which may be arranged randomly or blockwise, and in which a and b are in each case a number from 1 to 30, and d and e are in each case a number from 1 to 20, (O-A¹) and (O-D¹) are an ethyleneoxy unit, and (O-A²) and (O-D²) are a propyleneoxy unit, a butyleneoxy unit, a phenyloxirane unit, or mixtures of these.

25. An ethanolamine glyceryl ether condensation product as claimed in claim 17, wherein the radicals R¹, R² and R³ are identical and are a group of the formula (IIa), 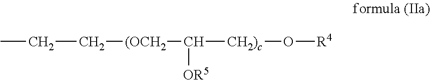
wherein
R⁵ is hydrogen,
c is a number from 1 to 25, and
R⁴ is hydrogen.

26. An ethanolamine glyceryl ether condensation product as claimed in claim 17, wherein the radicals R¹, R² and R³ are identical and are a group of the formula (IIa), 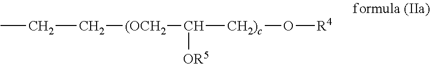
wherein
R⁵ is hydrogen,
c is a number from 1 to 25, and
R⁴ is a group of the formula (III)

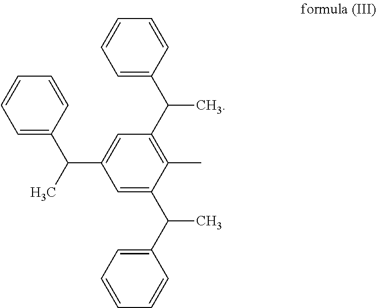

27. An ethanolamine glyceryl ether condensation product as claimed in claim 17, wherein the radicals R¹, R² and R³ are identical and are a group of the formula (IIb) 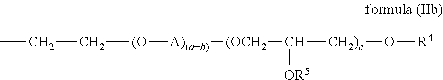
wherein
(O-A)_{(a+b)} is a uniform alkyleneoxy group and (a+b) is a number from 1 to 50 or is a mixed alkyleneoxy group of the formula -(O-A¹)ₐ-(O-A²)_{b}-, which can be arranged randomly or blockwise and in which (a+b) is a number from 1 to 50, (O-A¹) is an ethyleneoxy unit and (O-A²) is a propyleneoxy unit, a butyleneoxy unit, a phenyloxirane unit or mixtures of these,
R⁵ is hydrogen,
c is a number from 1 to 25, and
R⁴ is hydrogen.

28. An ethanolamine glyceryl ether condensation product as claimed in claim 17, wherein the radicals R¹, R² and R³ are identical and are a group of the formula (IIc) 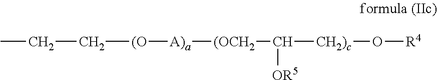
wherein
(O-A) is an alkyleneoxy group,
a is a number from 1 to 50,
R⁵ is hydrogen,
c is a number from 1 to 25, and
R⁴ is hydrogen.

29. An ethanolamine glyceryl ether condensation product as claimed in claim 17, wherein
R¹ is an alkyl group having 1 to 20, carbon atoms, and
R² and R₃ are identical and are a group of the formula (IId) 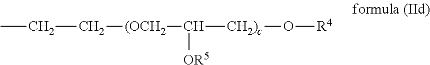
wherein
R⁵ is hydrogen,
c is a number from 1 to 25, and
R⁴ is hydrogen.

30. An ethanolamine glyceryl ether condensation product as claimed in claim 17, wherein
R¹ is an alkyl group having 1 to 10 carbon atoms, and
R² and R₃ are identical and are a group of the formula (IId) 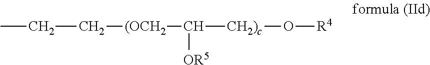
wherein
R⁵ is hydrogen,
c is a number from 1 to 25, and
R⁴ is hydrogen.

31. A dispersion comprising at least one ethanolamine glyceryl ether condensation products as claimed in claim 17.
